(19) United States
(12) Patent Application Publication
(10) Pub. No.: US 2024/0390318 A1
(43) Pub. Date: Nov. 28, 2024

(54) PHARMACEUTICAL COMPOSITION COMPRISING CDK INHIBITOR AND ID2 ACTIVATOR FOR PREVENTION OR TREATMENT OF BLADDER CANCER

(71) Applicants: THE ASAN FOUNDATION, Seoul (KR); University of Ulsan Foundation For Industry Cooperation, Ulsan (KR)

(72) Inventors: DONG MYUNG SHIN, Seoul (KR); YONGHWAN KIM, Seoul (KR)

(73) Assignees: THE ASAN FOUNDATION, Seoul (KR); University of Ulsan Foundation For Industry Cooperation, Ulsan (KR)

(21) Appl. No.: 18/695,887

(22) PCT Filed: Oct. 5, 2022
(86) PCT No.: PCT/KR2022/014930

§ 371 Date: Mar. 27, 2024

(30) Foreign Application Priority Data
Oct. 5, 2021 (KR) 10-2021-0131698

(51) Int. Cl. A61K 31/352; A61K 31/4709; A61P 35/00

(52) U.S. Cl. CPC A61K 31/352; A61K 31/4709; A61P 35/00

(57) ABSTRACT

The present invention relates to a pharmaceutical composition including a CDK inhibitor and an ID2 activator for the prevention or treatment of bladder cancer, wherein based on the finding that: among CDK1-TFCP2L1 pathway targets associated with urothelial differentiation in bladder cancer cells, ID2 is a factor significantly correlating with the tumor grade of bladder cancer; an ID2 activator exhibits a therapeutic effect on bladder cancer; and the combined administration of CDK1 inhibitor and ID2 activator induces apoptosis of bladder cancer cells and significantly suppresses the invasiveness of cancer cells, compared to administration thereof alone, thereby exhibiting a therapeutic effect of remarkably suppressing the size and progression of bladder cancer, a composition comprising a CDK inhibitor and an ID2 activator and a method for combined administration thereof are provided as novel therapeutic means for the prevention or treatment of bladder cancer.

### TECHNICAL FIELD

The present disclosure relates to a pharmaceutical composition for preventing or treating bladder cancer, including a CDK inhibitor and an ID2 activator.

### BACKGROUND ART

Bladder cancer (BC) is the 10^{th} most common cancer worldwide and a disease with high morbidity and mortality, with approximately 573,278 new cases of bladder cancer and more than 212,536 deaths in 2020. Bladder cancer is characterized by somatic mutations in a high proportion and has clinical and pathological heterogeneity.

Bladder cancer is broadly divided into non-muscle invasive bladder cancer and invasive bladder cancer depending on a degree of invasiveness. Non-muscle invasive bladder cancer is a lesion in which the cancer is confined to the mucous membrane without invasion into the muscle layer and may be treated relatively easily by injecting intravesical anticancer drugs or BCG depending on the presence of risk factors after performing transurethral resection of bladder tumor, but it becomes a matter when it comes to recurrence of cancer and progression to invasive cancer. On the other hand, invasive bladder cancer refers to a condition in which the cancer has infiltrated into the muscle layer, and its treatment may not only require radical cystectomy and complex urinary diversion, but also cause fatal consequences for the patient. Therefore, prediction on recurrence and progression after primary treatment, early detection, and prevention are very crucial.

Although various methods for diagnosis and treatment of bladder cancer have been developed, surgical treatments, chemotherapy drugs (methotrexate, vincristine, doxorubicin, cisplatin, and cytosine) and biologic therapies (Bacillus Calmette-Guerin, immune and inactivated bacteria solution) are being used in clinical practice as the treatment methods up to date, but treatment options are limited due to high cost, serious side effects, and various complications, as well as high recurrence and mortality rates of bladder cancer. Therefore, such frequent recurrence and progression of stage are frequently problematic in bladder cancer, and thus there is a need for the discovery of indicators that may effectively predict the recurrence of bladder cancer and progression into an invasive state, as well as development of treatment methods.

### DISCLOSURE OF THE INVENTION

### Technical Goals

An object of the present disclosure is to newly provide a method of preventing or treating bladder cancer, which may maximize a therapeutic effect by screening major factors for bladder cancer in order to discover effective new therapeutic targets for bladder cancer and evaluating whether the screened factors have a therapeutic effect on bladder cancer.

### Technical Solutions

The present disclosure provides a pharmaceutical composition for preventing or treating bladder cancer, including: a CDK inhibitor or a pharmaceutically acceptable salt thereof; and an ID2 activator or a pharmaceutically acceptable salt thereof as active ingredients.

In addition, the present disclosure provides a composition for co-administration for preventing or treating bladder cancer, including: a CDK inhibitor or a pharmaceutically acceptable salt thereof; and an ID2 activator or a pharmaceutically acceptable salt thereof as active ingredients.

### Advantageous Effects

According to the present disclosure, it was determined that ID2 is a factor that has significant relationship with a tumor grade of bladder cancer among targets of CDK1-TFCP2L1 pathway related to urothelial differentiation in bladder cancer cells, ID2 activator shows a therapeutic effect on bladder cancer, and combined administration of a CDK 1 inhibitor and an ID2 activator induces apoptosis in bladder cancer and remarkably suppresses the invasiveness of cancer cells compared to single administration of each of them to derive a therapeutic effect in which the size and progression of bladder cancer are remarkably inhibited, such that a composition including the CDK inhibitor and the ID2 activator and a co-administration method may be provided as a new therapeutic means for preventing or treating bladder cancer.

### BRIEF DESCRIPTION OF DRAWINGS

FIG. 1 shows a result of analyzing an expression level of ID2 as a therapeutic target for bladder cancer and relationship with bladder cancer using a TCGA data set. A red box represents tumor (T), and a grey box represents normal (N).

FIG. 2 shows a result of analyzing relationship of an expression level of ID2 according to a grade of bladder cancer using a TCGA data set.

FIG. 3 shows a result of analyzing relationship of an expression level of ID2 according to a pT category of bladder cancer using a TCGA data set.

FIG. 4 shows results of identifying expression for different target genes of CDK1-TFCP2L1 pathways other than ID2 in TCGA data cells of patients with bladder cancer.

FIG. 5 shows a result of evaluating expression levels of ID2, TFCP2L1, and CDK1 in bladder cancer cell lines 5637, HT1197, HT1376, and RT4.

FIG. 6 shows results of evaluating, via chromatin immunoprecipitation (ChIP) assay, whether TFCP2L1 affects transcription of ID2 in bladder cancer cell lines HT1197 and HT1376.

FIG. 7 shows results of evaluating an effect of TFCP2L1 deletion on ID2 expression in bladder cancer cell lines HT1197 and HT1376.

FIG. 8 shows a result of evaluating an effect of TFCP2L1 and ID2 expression on cell proliferation in bladder cancer cell lines.

FIG. 9 shows results of evaluating an effect of TFCP2L1 and ID2 expression on tumor sphere formation in bladder cancer cell lines.

FIG. 10 shows results of evaluating an effect of TFCP2L1 and ID2 expression on clonal genesis in bladder cancer cell lines.

FIG. 11 shows results of evaluating an effect of TFCP2L1 and ID2 expression on the invasiveness in bladder cancer cell lines.

FIG. 12 shows results of identifying growth and functional changes of bladder cancer in accordance with changes in expression values of ID2 in two groups of bladder cancer cells showing different patterns in ID2 expression.

FIG. 13 shows results of evaluating an effect of apigenin, an ID2 activator, on cell growth in bladder cancer cell lines.

FIG. 14 shows results of evaluating an effect of apigenin, an ID2 activator, on caspase-3 and poly-(ADP-ribose) polymerase (PARP) in bladder cancer cell lines.

FIG. 15 shows results of evaluating an effect of apigenin, an ID2 activator, on activation of apoptosis in bladder cancer cell lines.

FIG. 16 shows results of evaluating an effect of apigenin, an ID2 activator, on a tumor sphere forming ability and the invasiveness in bladder cancer cell lines.

FIG. 17 shows a method of administrating apigenin and RO-3306 to an orthotopic xenograft animal model induced with bladder cancer in order to evaluate an effect of administration of apigenin, an ID2 activator, and RO-3306, a CDK1 inhibitor, on bladder cancer.

FIG. 18 shows results of evaluating changes in tumor size upon apigenin administration in an animal model with bladder cancer.

FIG. 19 shows results of histological examination that evaluated changes in tumor grade upon apigenin administration in an animal model with bladder cancer.

FIG. 20 shows results of evaluating an effect of diosmetin, an ID2 activator, on bladder cancer cell lines.

FIG. 21 shows results of evaluating an effect of 4-hydroxychalcone, an ID2 activator, on bladder cancer cell lines.

FIG. 22 shows a result of analyzing an expression level of CDK1 as a therapeutic target for bladder cancer and relationship in bladder cancer using a TCGA data set. A red box represents tumor (T), and a grey box represents normal (N).

FIG. 23 shows a result of analyzing relationship of an expression level of CDK1 according to a grade of bladder cancer using a TCGA data set.

FIG. 24 shows results of analyzing relationship of CDK1 and ID2 expression using a TCGA data set.

FIG. 25 shows results of evaluating relationship of CDK1 and ID2 expression according to a pT category of bladder cancer.

FIG. 26 shows results of evaluating an effect on ID2 expression upon treatment of apigenin and RO-3306 in bladder cancer cell lines.

FIG. 27 shows results of identifying expression of ID2 upon treatment of CGP74514A and 4-hydroxychalcone in bladder cancer cell lines.

FIG. 28 shows results of evaluating effects on growth and apoptosis of bladder cancer cells upon treatment of apigenin and RO-3306 in bladder cancer cell lines.

FIG. 29 shows results of evaluating changes in tumor size upon administration of apigenin and RO-3306 in an orthotopic xenograft animal model induced with bladder cancer.

FIG. 30 shows results of histological examination that evaluated changes in a tumor grade upon administration of apigenin and RO-3306 in an orthotopic xenograft animal model induced with bladder cancer.

FIG. 31 shows results of evaluating effects on expression of ID2, CDK1, and TFCP2L1 proteins upon administration of apigenin and RO-3306 in an orthotopic xenograft animal model induced with bladder cancer.

### BEST MODE FOR CARRYING OUT THE INVENTION

The terms used herein are selected from general terms that are currently, widely used as much as possible in consideration of functions in the present disclosure, but they may vary depending on the intention or precedent of a person skilled in the art and the emergence of new technology. In addition, in certain cases, there are terms arbitrarily selected by the applicant, in which case their meaning will be described in detail in the corresponding description of the disclosure. Therefore, the terms used herein should be defined based on the meaning of the term and the overall content of the present disclosure, rather than simply the name of the term.

Unless otherwise defined, all terms used herein, including technical or scientific terms, have the same meanings as are generally understood by those skilled in the art to which the present disclosure pertains. Terms such as those defined in commonly used dictionaries should be construed as having meanings consistent with their meanings in the context of the relevant descriptions and should not be construed in an idealistic or excessively formal sense, unless clearly defined in the application.

A numerical range includes values defined in the above range. All maximum numerical limits given throughout the specification include all lower numerical limits, as clearly stated in the lower numerical limits. All minimum numerical limits given throughout the specification include all higher numerical limits, as clearly stated in the higher numerical limits. Any numerical limits given throughout the specification will include all better numerical ranges within a wider numerical range, as the narrower numerical limits are clearly stated.

Hereinafter, the present disclosure will be described in detail.

The present disclosure provides a pharmaceutical composition for preventing or treating bladder cancer, including a CDK inhibitor or a pharmacologically acceptable salt thereof; and an ID2 activator or a pharmaceutically acceptable salt thereof as active ingredients.

The CDK inhibitors may be, but are not limited to, RO-3306, CGP74514A, BEY-11707, ON-01500, R547, sodium oxamate, dinaciclib, BMS-265246, AZD5438, SU9516, riviciclib hydrochloride (P276-00), AT7519, and NU6027.

The ID2 activator may be, but are not limited to, apigenin, isoliquiritigenin, 4-hydroxychalcone, diosmetin, biochanin A, and luteolin.

The pharmaceutically acceptable salts refer to acid additive salts formed by pharmaceutically acceptable free acids, and the pharmaceutically acceptable salts refer to salts commonly used in the pharmaceutical industry, including, for example, inorganic ionic salts made from calcium, potassium, sodium, or magnesium and inorganic acid salts made from hydrochloric acids, nitric acids, phosphoric acids, bromic acids, iodic acids, perchloric acids, or sulfuric acids; organic acid salts made from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, or vanillic acid; sulfonates made from methanesulfonic acid, ethanesulfonic acid, benzenesulfonic acid, p-toluenesulfonic acid, or naphthalenesulfonic acid; amino acid salts made from glycine, arginine, and lysine; or amine salts made of trimethylamine, triethylamine, ammonia, pyridine, or picoline, but the type of salts referred in the present disclosure is not limited by these salts listed.

The pharmaceutical composition of the present disclosure may be prepared in the form of a unit volume by preparation using a pharmaceutically acceptable carrier in accordance with a method that may be easily carried out by those with ordinary knowledge in the art to which the present disclosure pertains, or it may be prepared by introducing in a multi-capacity container.

The pharmaceutically acceptable carriers are those commonly used in preparation and include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may additionally include lubricants, humectants, sweeteners, flavoring agents, emulsifiers, suspensions, and preservatives, in addition to the above ingredients.

As used herein, the content of the additives included in the pharmaceutical composition is not particularly limited and may be appropriately adjusted within the content range used in common preparation.

The pharmaceutical composition may be formulated in the form of one or more external agents selected from the group consisting of injectable formulations such as aqueous solutions, suspensions, and emulsions, pills, capsules, granules, tablets, creams, gels, patches, nebulizers, ointments, emplastrums, lotions, liniments, pastas, and cataplasmas.

The pharmaceutical composition of the present disclosure may include additional pharmaceutically acceptable carriers and diluents for formulation. The pharmaceutically acceptable carriers and diluents include, but are not limited to, excipients such as starch, sugars, and mannitol, fillers and extenders such as calcium phosphate, cellulose derivatives such as carboxymethylcellulose and hydroxypropyl cellulose, binders such as gelatin, alginate, and polyvinyl pyrrolidone, lubricants such as talc, calcium stearate, hydrogenated castor oil, and polyethylene glycol, disintegrating agents such as povidone and crospovidone, and surfactants such as polysorbate, cetyl alcohol, and glycerol. The pharmaceutically acceptable carriers and diluents may be biologically and physiologically friendly to subjects. Examples of diluents may include, but are not limited to, brine, water-soluble buffers, solvents, and/or dispersion media.

The pharmaceutical composition of the present disclosure may be administered orally or parenterally (e.g., applied intravenously, subcutaneously, intraperitoneally, or topically) depending on the desired method. When administered orally, it may be formulated as tablets, troches, lozenges, water-soluble suspensions, oily suspensions, preparation powders, granules, emulsions, hard capsules, soft capsules, syrups, or elixirs. When administered parenterally, it may be formulated as an injection solution, suppositories, powders for respiratory inhalation, aerosols for sprays, ointments, powders for application, oils, and creams.

The dosage of the pharmaceutical composition of the present disclosure may vary depending on the patient's condition and weight, age, sex, health status, dietary constitution specificity, nature of the preparation, severity of disease, administration time of the composition, method of administration, duration or interval of administration, excretion rate, and drug form, and it may be appropriately selected by a person skilled in the art. For example, it may range from about 0.1 to 10,000 mg/kg but is not limited thereby, and administration may be performed once to several times a day.

The pharmaceutical composition may be administered orally or parenterally (e.g., applied intravenously, subcutaneously, intraperitoneally, or topically) depending on the desired method. The pharmaceutical effective amount and effective dose of the pharmaceutical composition of the present disclosure may vary depending on the preparation method of the pharmaceutical composition, the mode of administration, the administration time and/or administration route, and a person with ordinary skill in the art may easily determine and prescribe the effective dose for the desired treatment. The administration of the pharmaceutical composition of the present disclosure may be performed once a day, or in several divided doses.

In addition, the present disclosure provides a composition for co-administration for preventing or treating bladder cancer, including a CDK inhibitor or a pharmaceutically acceptable salt thereof; and an ID2 activator or a pharmaceutically acceptable salt thereof as active ingredients.

The CDK inhibitor or pharmaceutically acceptable salt thereof; and the ID2 activator or pharmaceutically acceptable salt thereof may be prepared in a mixed form to be administered or prepared separately to be administered simultaneously or sequentially.

The CDK inhibitor or pharmaceutically acceptable salt thereof; and the ID2 activator or pharmaceutically acceptable salt thereof may be administered in a weight ratio of 1:10 to 1:15, specifically the CDK inhibitor may be administered via intraperitoneal injection in a dose of 1 mg/kg to 10 mg/kg, more specifically the CDK inhibitor RO-3306 or CGP74514A may be administered in a dose of 4 mg/kg, and the ID2 activator may be administered via intraperitoneal injection in a dose of 5 mg/kg to 150 mg/kg, and more specifically the ID2 activator apigenin, diosmetin, or 4-hydroxychalcone may be administered in a dose of 50 mg/kg.

### MODES FOR CARRYING OUT THE INVENTION

Hereinafter, to help understanding of the present disclosure, experimental examples and examples will be described in detail. However, the following experimental examples and examples are merely illustrative of the contents of the present disclosure, and the scope of the present disclosure is not limited to the following experimental examples and examples. The experimental examples and examples of the present disclosure are provided to more completely explain the present disclosure to those of ordinary skill in the art.

### <EXPERIMENTAL EXAMPLE>EXPERIMENTAL MATERIALS AND METHODS

The following experimental examples are intended to provide experimental examples that are commonly applied to each example according to the present disclosure.

1. Clinical Cohort Analysis on Patients with Bladder Cancer (BC)

Two independent clinical cohorts for patients with bladder cancer (BC) were used in The Cancer Genome Atlas (TCGA; https://cancergenome.nih.gov). The first cohort is about 131 patients with high-risk muscle-invasive bladder carcinomas (MIBC), and the second is about a multiplatform analysis for 412 patients with MIBC. Clinicopathological features including survival, tumor stage and grade as well as gene expression datasets were obtained from the UCSC Xenia project (http://xena.ucsc.edu/). Gene Expression Profiling Interactive Analysis (GEPIA; http://gepia.cancerpku.cn/index.html) was used to analyze outcomes of tumor/non-tumor differential expression and pairwise correlated gene expression, which is an interactive web-based tool that provides fast, customizable functionality based on TCGA and Genotype Tissue Expression (GTEx; https://www.gtexportal.org/) datasets. In the second TCGA study for BC patients, a Kaplan-Meier survival analysis based on high (red) and low (black) expression levels of the ID family gene was performed using Prism 7.0. Gene expression datasets from this TCGA cohort were used for differential or paired gene expression analysis based on tumor stage and grade.

### 2. Cell Culture

Human BC cell lines T24, 5637, HT1197, HT1376, and RT4 were cultured in Eagle's Minimum Essential (for HT1197 and HT1376), McCoy's 5a Medium Modified (for T24 and RT4), and RPMI-1640 (for ATCC37) medium (ATCC, Manassas, VA, USA) including 10% heat-inactivated FBS (Hyclone, Pittsburgh, PA, USA) and penicillin/streptomycin (Cellgro, Pittsburgh, PA, USA). 40 μM apigenin (Sigma-Aldrich, Burlington, MA, USA), 4-hydroxychalcone (Sigma-Aldrich), and diosmetin (Sigma-Aldrich) were treated for 24 hours to activate ID2 expression, and 20 μM RO-3306 (Sigma-Aldrich) and CGP74514A (Sigma-Aldrich) were treated for 24 hours to inhibit CDK1 activity.

### 3. Ectopic Expression and Gene Silencing

To induce ectopic expression or silencing of the target gene, the corresponding open reading frame (ORF) or specific shRNA construct was cloned into pLEX307 (Addgene, plasmid #41392) and pLenti6/Block-iT lentivrial vector (Invitrogen, Waltham, MA, USA), respectively. For the human ID2 ORF, the pDONR223_ID2_WT_V5 was used as Jesse Boehm & Matthew Meyerson & David Root (Addgene plasmid #82960). Lentivirus was produced using a four-plasmid transfection system (Invitrogen) and concentrated using the Lenti-X Concentrator kit (Clontech, Mountain View, CA, USA). Gene expression and functional analysis were performed on the fourth day after lentivirus infection. Information on each ORF and target sequence of each shRNA is shown in Tables 1 and 2 below.

TABLE 1

| ORF constructs | Source | Identifier |
|---|---|---|
| pBluescriptR_human | Dharmacon | MHS6278- |
| TFCP2LI | | 202806269 |
| pDONR223 human | Addgene | 82960 |
| ID2 | | |

TABLE 2

| Oligonucleotides | |
|---|---|
| (shRNA) | Sequence |
| human TFCP2L1 | GCTCTTCAACGCCATCAAAGG |

### 4. Analysis on Chromatin Immunoprecipitation (ChIP) Assays and Gene Expression

HT1197 and HT1376 BC cells were transfected with human TFCP2L1 ORFs that are cloned in pCMV_3Tag-1 vector (Agilent Technologies, Santa Clara CA, USA) using Lipofectamine 2000 (Invitrogen). After 24 hours of transfection, the isolated cross-linked chromatin was cleaved in the cell extract (1×10⁷ cells) using the Bioruptor Plus sonication device (Diagenode Inc., Denville, NJ, USA) under a standard setting (4 cycles with a 20-sec pulse condition having a 30-sec rest interval on ice). ChIP assay was performed using Magna ChIP G kits (Millipore, Billerica, MA, USAA).

Real-time quantification PCR was used to perform quantitative analysis of gene expression. Total RNA was extracted using the QIAGEN RNeasy RNA isolation kit (QIAGEN, Valencia, CA, USA), and 50 ng of total RNA was reverse transcribed using Taqman Reverse Transcription Reagents (Applied Biosystems, Foster City, CA, USA). Threshold cycles (Ct) were used to determine the relative expression levels of target genes using the 2-DDCt method. The expression of GAPDH was used as an endogenous control gene. Primers used in ChIP and gene expression analysis are shown in Tables 3 and 4 below.

TABLE 3

| Oligonucleodes | | |
|---|---|---|
| (qPCR) | Forward primer | Reverse primer |
| human TFCP2LI | GCTCTTCAACGCCA | CAGGGGCACTCGAT |
| | TCAAA | TCTG |
| human ID2 | CAGCATCCCCCAGA | CGATCTGCAGGTCC |
| | ACAAGAA | AAGATGT |

TABLE 4

| Oligonucleodes | | |
|---|---|---|
| (qChIP) | Forward primer | Reverse primer |
| human ID2_#1 | CTCCGATGGGTTGC | CGGCAGCTCTAAAA |
| | AGTGAA | TCACAGCTA |
| human ID2_#2 | TGCAGCACGTCATC | CTGGTGATGCAGGC |
| | GACTACAT | TGACAA |

### 5. Western Blot Analysis

Cell extracts (30 μg) were prepared in RIPA lysis buffer (Santa Cruz Biotechnology) supplemented with a protease and phosphatase inhibitor cocktail (Roche, Indianapolis, IN, USA) and isolated in a 12% SDS-PAGE gel. An expression level of the labeled protein was assessed by probing with the following antibodies: ID2 (NBP-88630; Novusbio, Centennial, CO, USA), TFCP2L1 (OAAB09732; Aviva Systems Biology, San Diego, CA, USA), CDK1 (sc-54; Santa Cruz), PARP (9542; Cell Signaling), Cleaved Caspase-3 (9661; Cell Signaling), b-ACTIN (A5441; Sigma-Aldrich), and Flag-epitope (F3165; Sigma-Aldrich).

### 6. Immunocytochemistry Assay

For immunocytochemistry, human BC cells fixed with 4% paraformaldehyde (Sigma-Aldrich) were stained with antibodies specific to ID2 (NBP-88630; Novusbio) or TFCP2L1 (OAAB09732; Aviva Systems Biology) and visualized using Alexa 488-conjugated anti-rabbit antibody (A11008, Molecular Probes, Grand Island, NY, USA). The stained samples were photographed using an inverted fluorescence microscope (EVOS FL Color Imaging System, Life Technologies, Carlsbad, CA, USA).

### 7. Analysis on Cell Proliferation and Apoptosis

The cell proliferation ability was determined by MTT assay (Sigma-Aldrich). Apoptosis was analyzed by annexin-V fluorescein isothiocyanate (FITC)/propidium iodide (PI) assay. Cells were subjected to trypsin, harvested, washed with PBS, resuspended in annexin-V binding buffer (10 mM HEPES, pH 7.4, 140 mM NaCl, 2.5 mM CaCl₂)), and labeled with Annexin-V FITC and PI. FITC- and/or PI-labeled cell colonies were quantified by flow cytometry (Beckman Coulter, Brea, CA, USA).

### 8. Tumor Sphere Formation and Limiting Dilution Assay

To form tumor spheres, BC cells were resuspended in a single-cell suspension mixed with serum-free keratinocyte growth medium (Gibco, Waltham, MA, USA) and growth factor-reduced Matrigel (BD Biosciences, Mountain View, CA, USA) in a 1:1 ratio, and cultured on ultra-low attachment plates (Costar, Corning, NY, USA). The size of the tumor sphere was measured for 7 days after the first culture. For quantitative analysis, Image J software (National Institute of Mental Health, Bethesda, MD, USA) was used to measure the circumference of tumor sphere in eight representative regions randomly selected from each group.

For the limiting dilution assay, BC cells were diluted to a density of 1 cell per well and cultured in 50 μL of culture medium. The smeared cells were cultured up to 10 days with a new medium added every 2 days, and the colony count was calculated for quantitative analysis.

### 9. Analysis on In Vitro Cell Invasion

The upper chamber of the Transwell permeable support (Corning Inc, Corning, NY, USA) with an 8.0 μm pore polycarbonate membrane filter was coated with Matrigel (BD Biosciences) diluted in a 1:5 ratio. BC cells were loaded into the upper chamber at 2×10⁴ cell/well concentrations in 100 μL of serum-free DMEM, and culture medium containing 3% FBS was filled into the lower chamber. Cell invasiveness was evaluated by culturing cells at 37° C. for 24 hours in a 5% CO₂ incubator and then counting the number of cells that migrated to the bottom of the membrane. Three fields of view (magnification, ×200) were randomly selected from each transwell chamber for quantitative analysis.

### 10. Orthotopic Implantation (Xenograft) of BC Cells

All animal experiments conducted in this experiment were carried out with the approval of the Institutional Animal Care Committee (IACUC-2020-12-209) of the University of Ulsan College of Medicine. 8-week-old male NOD/ShiLtJ-Prkdc^{em1AMC}I12rg^{em1AMC} (NSGA) mice were purchased from GEM Biosciences Inc. (Cheongju, South Korea). 100 μl of 1.0×10⁶ HT1376 BC cells were injected into the anterior wall of the bladder and the outer layer of the dome using a 500 μm syringe and a 26-gauge needle in the mice adapted for 1 week at the Animal Laboratory of Asan Medical Center, Seoul. Three weeks after orthotopic transplantation of BC cells, mice were injected intraperitoneally with RO-3306 (4 mg/kg) and apigenin (50 mg/kg), either alone or in combination, for 6 times at 4-day intervals. Mice and injection sites were monitored every 2 days for 45 days after the initial administration of BC cells. Tumor size was measured at the endpoint, and the tumor site was recovered by incision to perform histological examination or immunofluorescence assay. Mice were randomly assigned to the treatment group (N=5 or 10) and randomized to the sequence of cell transplantation, treatment, evaluation, and daily checkup. Investigations related to tumor size measurement and histological assessment were conducted as blind tests for the treatment group.

### 11. Histological Examination

For histological analysis, the bladder of xenograft mice was fixed with 4% paraformaldehyde for 1 day. After 24 hours of cryoprotection in 30% sucrose, each bladder was cut into 20 μm sections using cryostat (Leica, Lussloch, Germany) and stained with hematoxylin and eosin (H&E). For immunofluorescence (IF) staining, the bladder was stained with antibodies specific to ID2 (NBP-88630; Novusbio), TFCP2L1 (OAAB09732; Aviva Systems Biology), CDK1 (ab131450; Abcam, Cambridge, MA, USA), CD44 (ab78960; Abcam), and Cytokeratin 14 (KRT14; Ab7800). Alexa Fluor 488-conjugated (A11001 and A11008) anti-mouse and anti-rabbit antibodies or Alexa Fluor 546-conjugated anti-rabbit antibodies (A11010) were used as secondary antibodies (molecular probes). Nuclei were counterstained with 4′,6-diamino-2-phenylindole (DAPI; D9542; Sigma-Aldrich). Three representative areas were randomly selected per slide. The stained samples were photographed using an inverted fluorescence microscope (EVOS FL Color Imaging System, Life Technologies).

### 12. Statistics

Quantitative outcomes were statistically analyzed using one- or two-way ANOVA in combination with the non-parametric Mann-Whitney test or the Bonferroni post hoc test. All analyses were performed using GraphPad Prism 7.0 software (GraphPad Software, La Jolla, CA, USA), and p<0.05 was considered statistically significant.

### Example 1. Screening of Therapeutic Targets in Bladder Cancer (BC) Cells

It has been reported that ectopic expression of TFCP2L1 including TFCP2L1 silence or Thr177 phosphorylated null mutation induces expression of differentiation genes including BMP, GATA, and ID families that stimulate urothelial differentiation. In addition, pharmacological activation of the BMP pathway by low dose FK506 has been reported to be effective in inhibiting progression in 70-80% of patients with non-muscle invasive urothelial carcinoma at initial diagnosis. The above report suggests that the ID gene may be a clinically important factor in bladder cancer (BC).

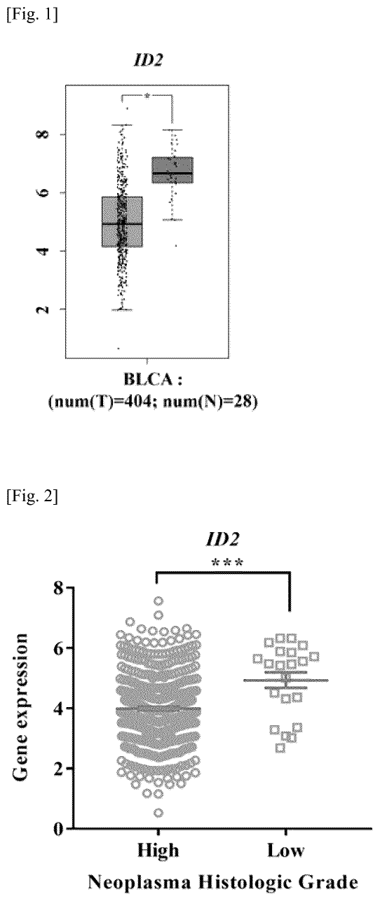

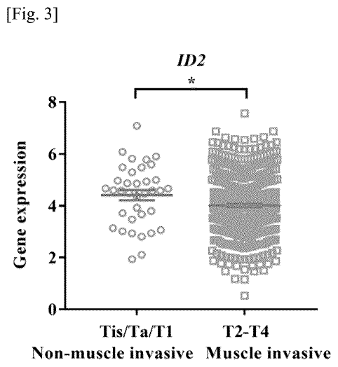

In order to discover major factors of bladder cancer among the ID families, as a result of analyzing the TCGA dataset of BC patients using the GEPIA and UCSC Xena Project (http://xena.ucsc.edu/) web servers, according to FIG. 1, it was shown that the expression of ID2 among the ID families was higher in bladder cancer than in the normal urothelium. In addition, as a result of comparing the tumor grade according to an expression level of ID2 for BC patients in a TCGA data set, according to FIG. 2, it was found that the higher the tumor grade, the more the expression of ID2 was suppressed. In particular, according to FIG. 3, ID2 transcriptome showed a tendency to significantly decrease in patients with bladder cancer above pT2 where muscle invasion was observed in the tumor stage in the pT category indicating muscle invasion of the bladder.

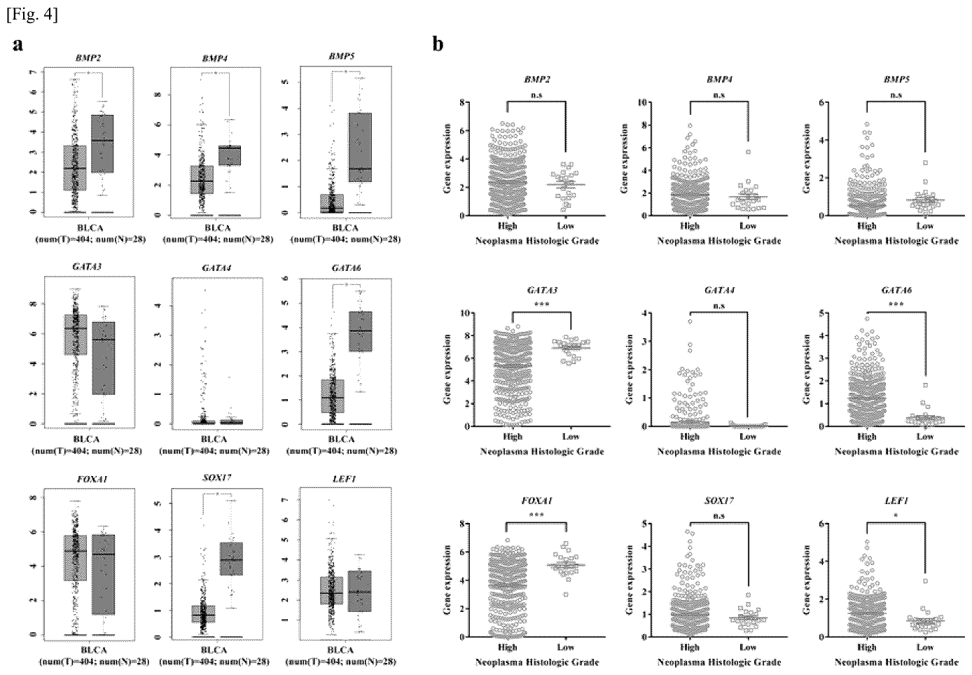

In addition, as a result of analyzing the expression of BMP family and GATA family, which are urothelial differentiation genes identified with ID2 in the CDK1-TFCP2L1 pathway in the TCGA dataset of bladder cancer patients, according to FIG. 4, the expression of the BMP family was low in bladder cancer, with no significant difference compared by high-low according to bladder cancer grade, whereas in GATA family, only GATA6 showed significant reduction in expression in bladder cancer with a significant decrease shown in low-grade bladder cancer. The above results prove that, among the urothelial differentiation genes identified in the CDK1-TFCP2L1 pathway, cancer cells and normal cells in the bladder show significant differences in gene expression, and ID2, which showed low expression in the high-risk grade, is the key factor.

### Example 2. Evaluation of ID2 as Therapeutic Target in Bladder Cancer (BC) Cells

Endogenous expression levels of ID2 and TFCP2L1 in BC cells with different molecular classification functions were compared with various molecular classification features, including base-like subtypes (5637 and HT1197) of muscle-invasive BC (MIBC), lumen-like subtypes (HT1376), mixed subtypes (T24 and UMUC3), and a RT4 cell line which is a model of non-muscle invasive BC. In most BC cell lines, ID2 and TFCP2L1 transcript levels show contradictory tendencies, and as a result of analyzing these inter-expression levels by Western blot and immunofluorescence staining assays, according to FIG. 5, they showed a marked opposite tendency at a protein level. In particular, ID2 protein was rarely detected in the HT1197 and HT1367 BC cell lines, which had the highest TFCP2L1 protein content.

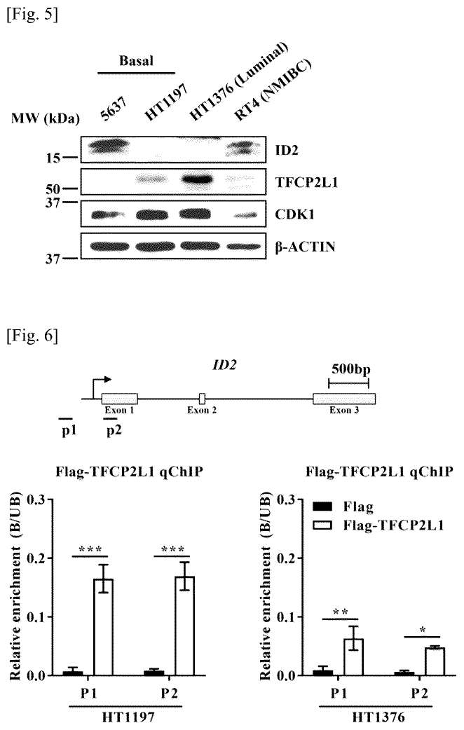

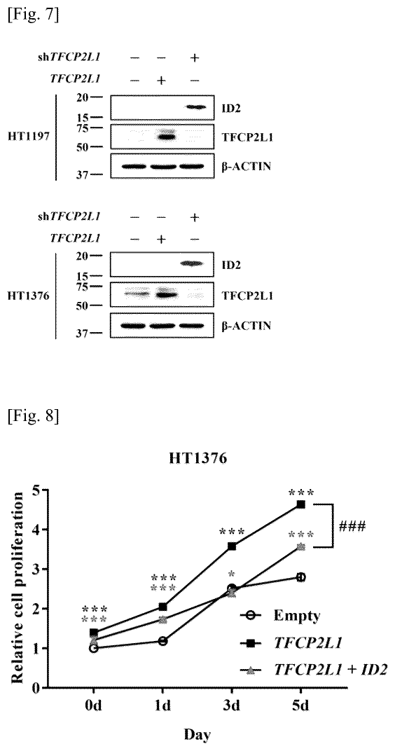

To determine whether TFCP2L1 directly inhibits transcription of ID2, chromatin immunoprecipitation (ChIP) assay was performed on HT1197 and HT1367 BC cells including flag-tagged TFCP2L1 (Flag-TFCP2L1) proteins. As a result of ChIP-qPCR assay, according to FIG. 6, it was found that the TFCP2L1 binds to the promoter of the ID2 locus in the BC cell line. Furthermore, according to FIG. 7, as it was observed that expression of ID2 is inhibited by the silence of the TFCP2L1, the results demonstrate that ID2 is a direct target of inhibition for TFCP2L1 in BC cells.

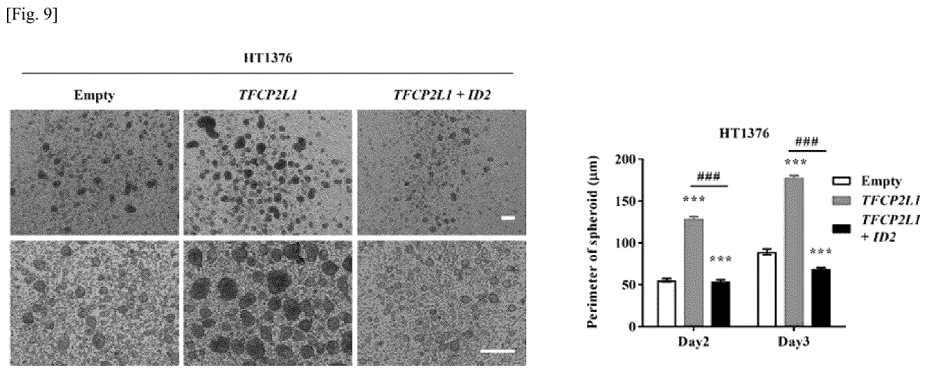

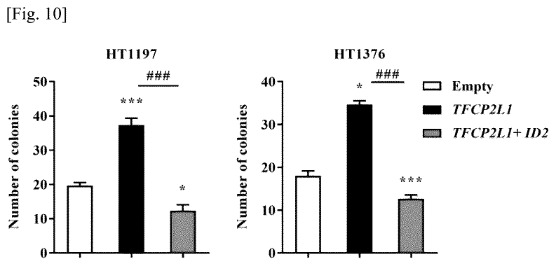

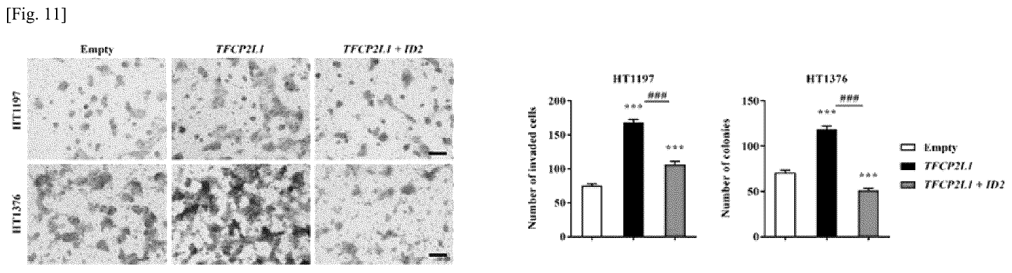

Whether ID2 affects cell proliferation and stemness of BC cells by the CDK1-TFCP2L1 pathway was evaluated. According to FIG. 8, enhanced expression of ID2 in HT1376 and HT1197 BC cells was found to inhibit stimulation of cell proliferation by TFCP2L1 overexpression. As a result of investigating the stemness of BC cells by tumor sphere and clonal genesis, according to FIG. 9, it was found that tumor sphere formation increased by TFCP2L1 overexpression was inhibited by enhanced expression of ID2. Further, according to FIG. 10, the negative outcome of ID2 for TFCP2L1-induced self-regenerating activity was observed in clonal generation limiting dilution assay. In addition, according to FIG. 11, trans-well chamber analysis showed that HT1376 and HT1197 BC cells with TFCP2L1 overexpression showed the higher invasiveness compared to control cells, but the invasiveness was impaired when ID2 was stimulated. The results demonstrate that ID2 is a target factor that inhibits the TFCP2L1 to regulate the stemness and invasiveness of BC cells.

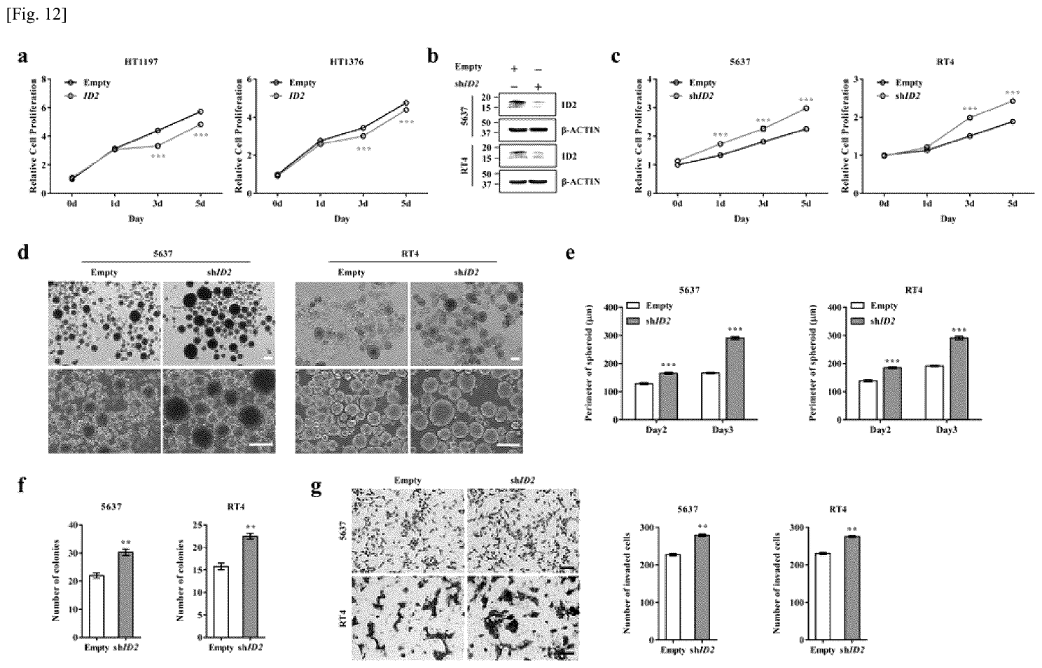

Furthermore, changes in growth and functions of bladder cancer in accordance with variation in expression values of ID2 were identified in two groups of bladder cancer cells with different ID2 expression patterns. As a result, according to FIG. 12, it was found that overexpression of ID2 in bladder cancer cells (HT1197, HT1376) with weak expression of ID2 inhibited cell proliferation, while inhibition of ID2 expression in bladder cancer cells (5637, RT4) with high expression of ID2 increased cell proliferation. When ID2 expression was reduced, changes in the function of bladder cancer cells (tumorigenesis, invasiveness) were identified. Both bladder cancer cells (5637, RT4) with reduced ID2 expression showed an increase in tumor growth size and number. In addition, the invasiveness was also found to be increased in two bladder cancer cells with reduced expression of ID2. Therefore, the suppression of ID2 expression enhanced the growth of bladder cancer cells and the overall function of cells, indicating that ID2 is a therapeutic target in bladder cancer (BC).

### Example 3. Evaluation of Therapeutic Ability of Bladder Cancer (BC) Upon ID2 Activation

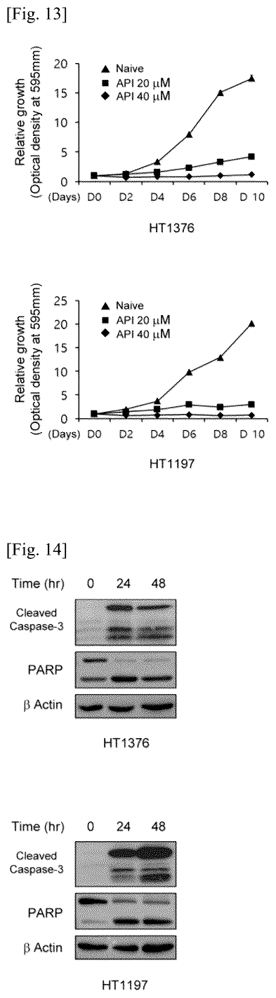

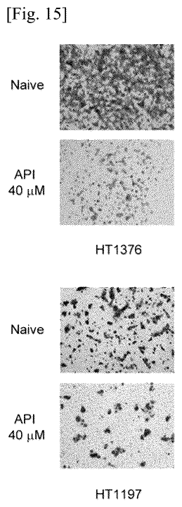

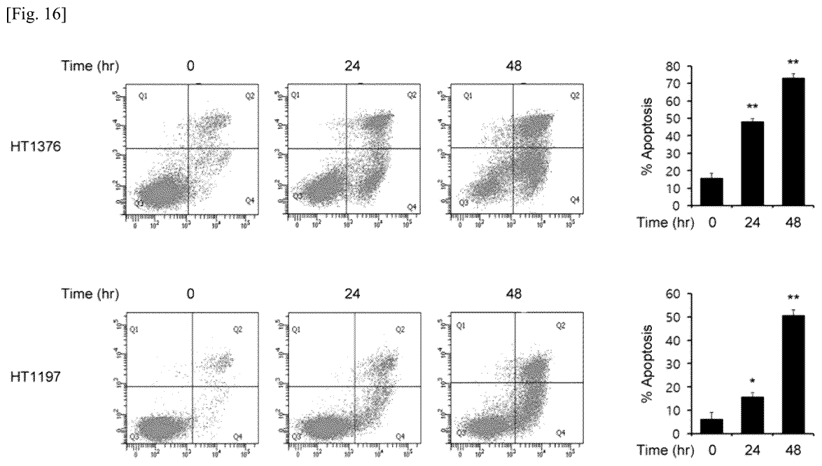

To validate whether ID2 activation may be a therapeutic target for bladder cancer, BC cells were treated with apigenin, a non-toxic dietary flavonoid that activates BMP signaling and ID2 induction, to assess whether changes in cell growth, a degree of apoptosis, and cell invasiveness occur. According to FIG. 13, it was found that the growth of BC cells decreased significantly as a dosage of apigenin increased. In addition, according to FIG. 14, treatment of BP cells with apigenin activators resulted in cleavage of caspase-3 and poly-(ADP-ribose) polymerase (PARP). According to FIG. 15, as a result of treating apigenin in a time-dependent manner, it was found that apoptosis of BC cells was activated. In addition, according to FIG. 16, apigenin was found to remarkably inhibit the tumor sphere forming ability and invasiveness of BC cells. The above results prove that apigenin, an ID2 activator, may be a new therapeutic agent for bladder cancer.

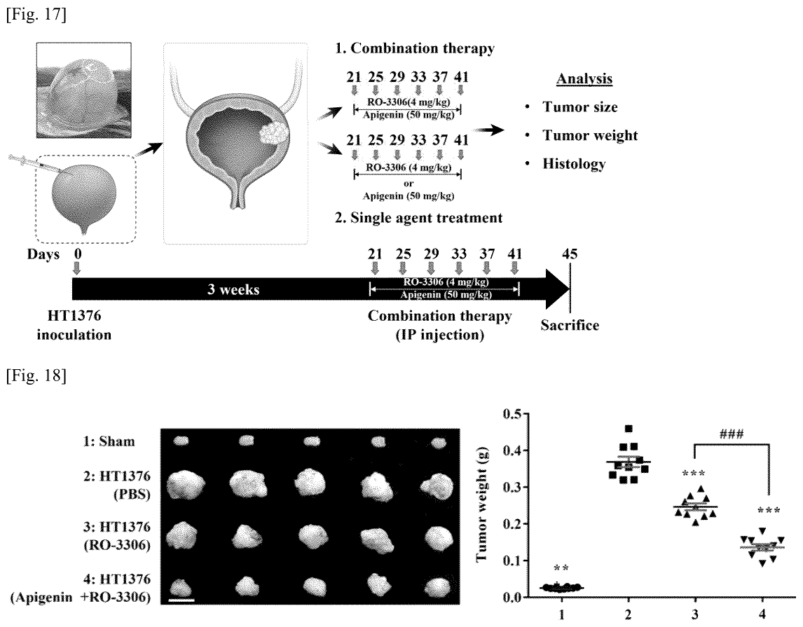

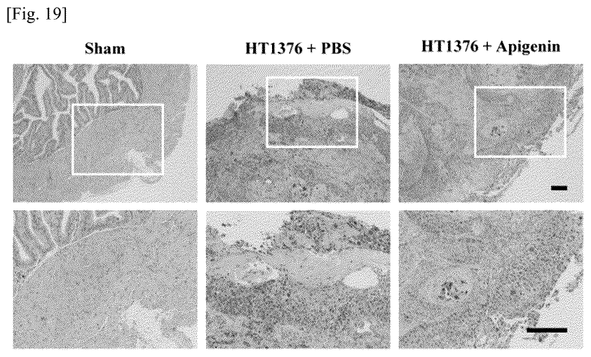

To evaluate whether the therapeutic effect of the apigenin on bladder cancer is seen in vivo, evaluation was made on whether the therapeutic effect is derived by administrating apigenin to an orthotopic xenograft BC animal model in which HT1376 cells were transplanted into the outer bladder layer of NSG immunodeficient mice. According to FIG. 17, apigenin was injected into the abdominal cavity in animal models six times at 4-day intervals. According to FIG. 18, tumor growth was suppressed by 74±6.13% in the group administered with apigenin. In addition, according to FIG. 19, histological examination showed that there was a therapeutic effect of apigenin on bladder cancer regardless of tumor grade. The results demonstrate that apigenin, the ID2 activator, exerts antitumor effects alone in bladder cancer.

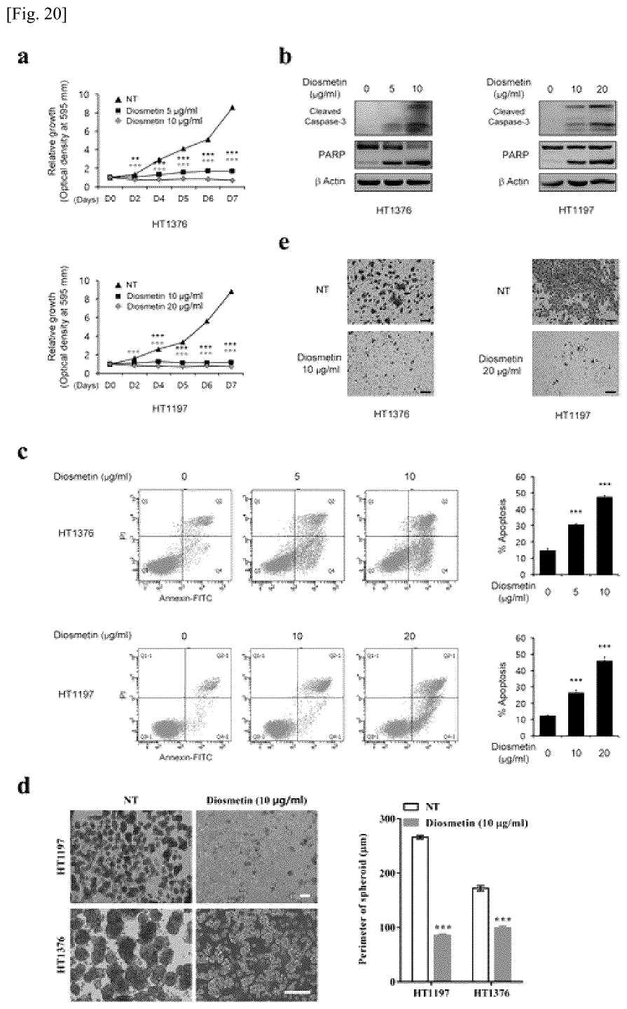

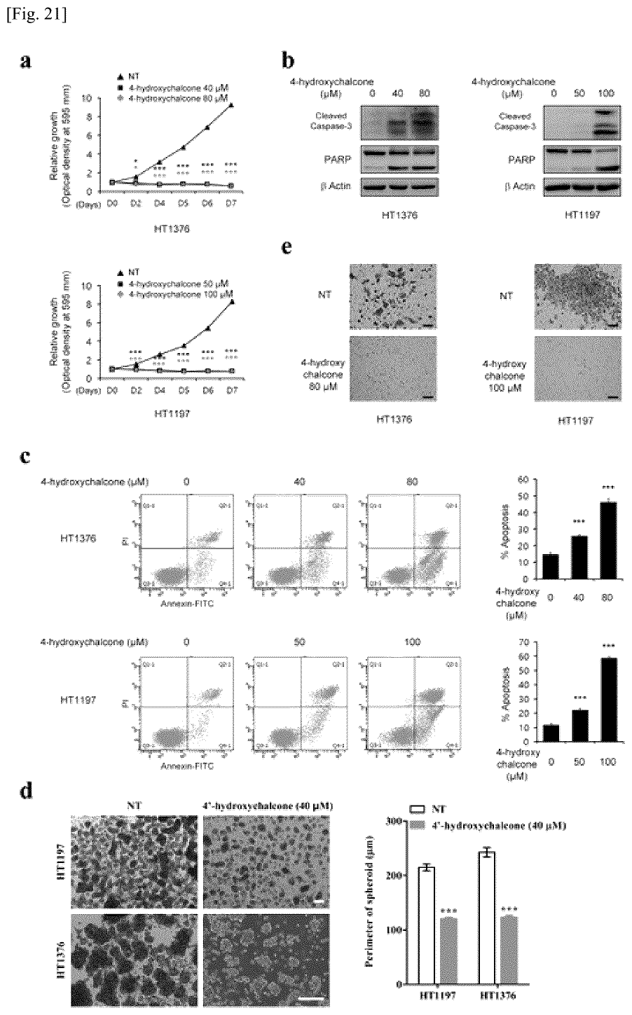

Since apigenin mediates anticancer activity not only through ID2 but also through multiple signaling pathways, in order to find once more that these results are the outcome of ID2 activity, investigation was made on whether similar outcomes as apigenin are derived using diosmetin or 4-hydroxychalcone, which are substances that activate the expression of other ID2 proteins. As a result, according to FIG. 20, it was found that diosmetin reduced bladder cancer cell growth, activated apoptosis, and decreased tumor forming ability similar to apigenin. Furthermore, according to FIG. 21, it was observed that 4-hydroxychalcone also reduced the growth of bladder cancer cells and activated apoptosis, similar to apigenin. In addition, tumor forming ability was reduced.

Therefore, ID2 activators, including apigenin, were found to have an anti-cancer effect on bladder cancer.

### Example 4. Evaluation on Importance of Cross-Expression of CDK1 and ID2 in Bladder Cancer (BC)

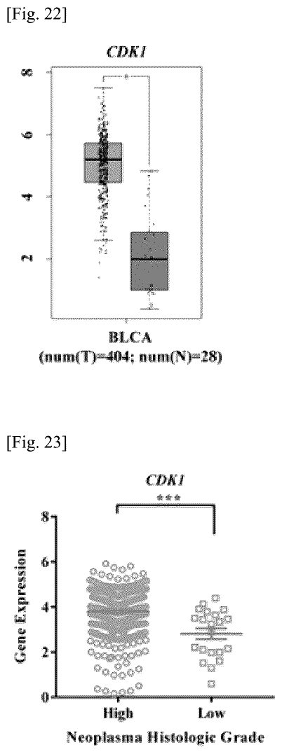

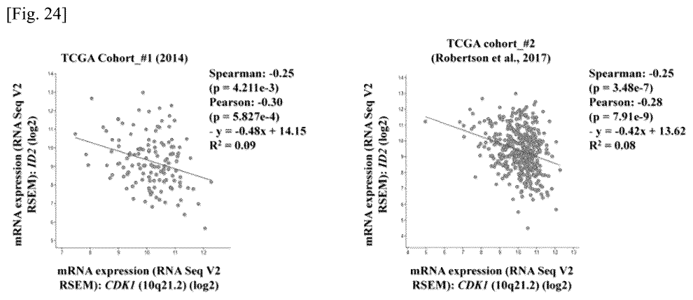

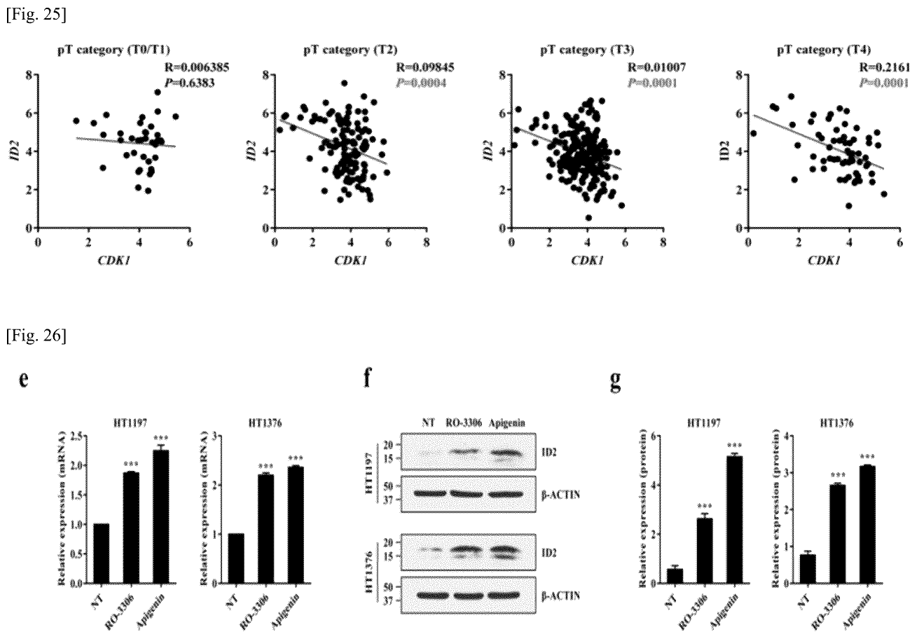

Evaluation was made on whether expression of CDK1, an upstream activator of TFCP2L1, affects an antitumor effect of apigenin. The expression of CDK 1 and its relationship with ID2 were evaluated in the TCGA cohort. According to FIG. 22, the expression of CDK1 was higher in bladder cancer than in normal urinary tract epithelial tissues. According to FIG. 23, consistent with ID2 expression, the higher the level of CDK1 transcript in the tumor grade of bladder cancer, the higher the tendency for CDK1 expression. Furthermore, according to FIG. 24, high levels of CDK1 in two independent TCGA cohorts were found to have relationship with decreased ID2 expression. According to FIG. 25, reverse expression on CDK1 and ID2 was found to have relationship with the tumor stage in the pT category. The above results demonstrate that potential association of CDK1 and ID2 is clinically important in patients with bladder cancer.

### Example 5. Increase in ID2 Expression by CDK Inhibitors

To validate whether CDK inhibition may be a therapeutic target for bladder cancer, it was determined whether treatment of BC cells with RO-3306, the CDK1 inhibitor, increases ID2. As a result, according to FIG. 26, when two types of bladder cancer cells (HT1197 and HT1376) were treated with CDK1 inhibitor (RO-3306) and apigenin respectively, the expression of ID2 was increased at the RNA and protein levels, similar to apigenin in both bladder cancer cells.

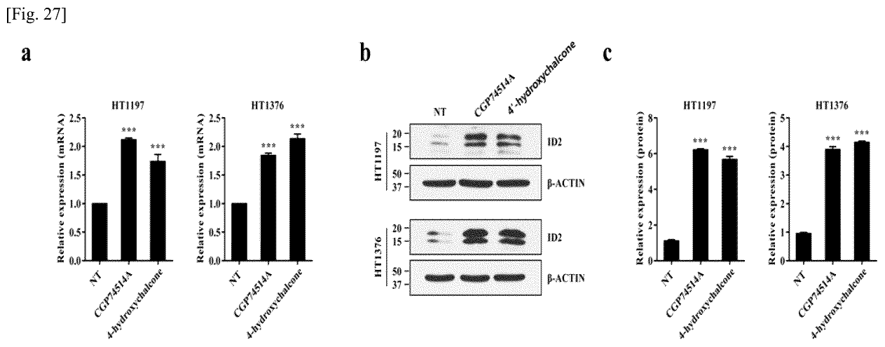

In addition, ID2 expression was re-checked with another CDK1 inhibitor (CGP74514A) that inhibits the expression of CDK1 in order to find out again whether the increase in ID2 in RO-3306-treated bladder cancer cells was due to suppression of the CDK1 expression. As a result, according to FIG. 27, an increase in ID2 expression was observed in the other CDK1 inhibitor (CGP74514A) and another ID2 activator (4-hydroxychalcone) similar to the outcome of RO-3306.

These results prove the potential association between CDK1 and ID2 in bladder cancer.

### Example 6. Evaluation on a Therapeutic Effect of Apigenin and RO-3306 in Bladder Cancer

Based on clinical findings on association of CDK1 and ID2 and outcomes of cell-model experiments of increased ID2 expression by CDK inhibitors, evaluation was conducted whether administration of CDK1 inhibitors alone or in combination with apigenin in bladder cancer cells and animal models has the therapeutic effect on bladder cancer. Evaluation was made on whether combination of apigenin and RO-3306, the CDK1 inhibitor, has a synergistic effect in the treatment of bladder cancer.

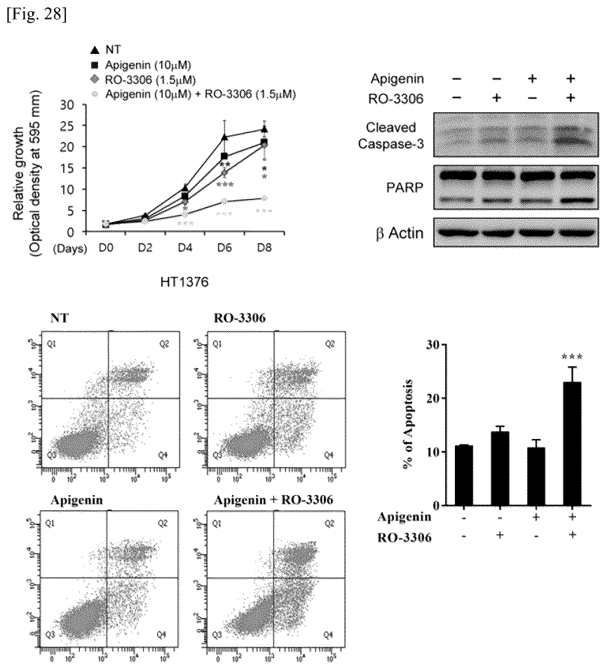

According to FIG. 28, single treatment with apigenin or RO-3306 at low concentrations had no significant effect on cell growth, proliferation and cleavage of caspase-3 and PARP, whereas co-administration of apigenin and RO-3306 induced caspase-3 and PARP cleavage. The above results demonstrate that treatment with apigenin or RO-3306 alone does not induce proliferation and apoptosis of bladder cancer cells, but co-administration of apigenin and RO-3306 induces inhibition in the proliferation of bladder cancer cells and apoptosis.

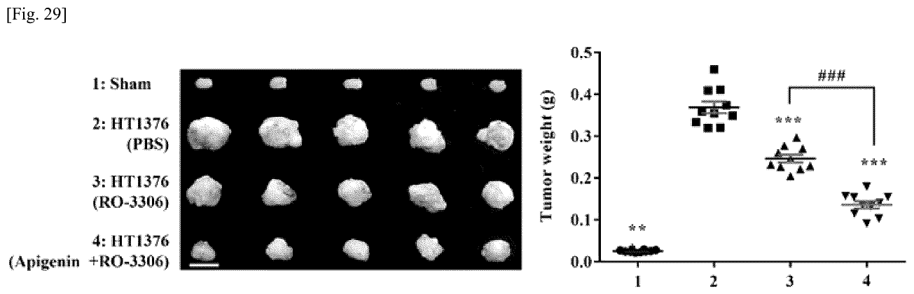

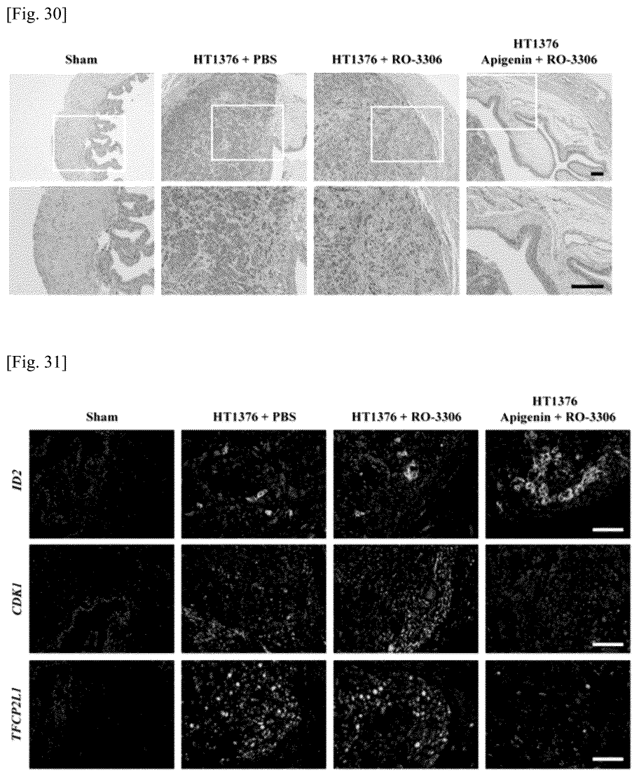

To evaluate whether the bladder cancer therapeutic effect due to administration of RO-3306 alone or in combination with apigenin was also seen in vivo, administration of RO-3306 alone or in combination with apigenin was performed to an orthotopic xenograft BC animal model in which HT1376 cells were implanted in the outer bladder layer of NSG immunodeficient mice. According to FIG. 29, tumor growth was found to be inhibited by 66.8±2.48% and 36.9±2.31% respectively in single administration of RO-3306 and co-administration with apigenin. Histological analysis was performed to validate whether co-administration of RO-3306 and apigenin exhibited antitumor effects in xenograft models for bladder cancer. As a result, according to FIG. 30, the size and progression of bladder cancer were remarkably inhibited by the co-administration of RO-3306 and apigenin. In addition, according to FIG. 31, the expression level of ID2 protein was low while CDK1 and TFCP2L1 proteins were high in the control group and RO-3306 single administration group, but the expression of ID2 protein was high while expression of CDK1 and TFCP2L1 proteins was suppressed in the co-administration of RO-3306 and apigenin.

The results above showed that the CDK1-targeted inhibitor enhanced the anti-cancer effect of apigenin which was the ID2 activator, and proved that the co-administration of the CDK1-target inhibitor and the ID2 activator showed a synergistic effect compared to the single administration of each of them.

While specific parts of the present invention have been described in detail above, it is clear to those skilled in the art that these specific descriptions are merely preferred example embodiments and do not limit the scope of the present disclosure. In other words, the substantial scope of the present disclosure is defined by the appended claims and their equivalents.

1. A method of preventing or treating bladder cancer, comprising:
administering a pharmaceutical composition comprising a CDK inhibitor or a pharmaceutically acceptable salt thereof; and an ID2 activator or a pharmaceutically acceptable salt thereof as active ingredients to a subject.

2. The method of claim 1, wherein the CDK inhibitor is any one or more selected from the group consisting of RO-3306, CGP74514A, BEY-11707, ON-01500, R547, sodium oxamate, dinaciclib (SCH727965), BMS-265246, AZD5438, SU9516, riviciclib hydrochloride (P276-00), AT7519, and NU6027.

3. The method of claim 1, wherein the ID2 activator is any one or more selected from the group consisting of apigenin, isoliquiritigenin, 4-hydroxychalcone, diosmetin, biochanin A, and luteolin.

4. A method of preventing or treating bladder cancer, comprising:
co-administration a composition comprising a CDK inhibitor or a pharmaceutically acceptable salt thereof; and an ID2 activator or a pharmaceutically acceptable salt thereof as active ingredients to a subject.

5. The method of claim 4, wherein the CDK inhibitor or pharmaceutically acceptable salt thereof; and the ID2 activator or pharmaceutically acceptable salt thereof are prepared in a mixed form to be administered or prepared separately to be administered simultaneously or sequentially.

6. The method of claim 4, wherein a dosage of the CDK inhibitor is 1 mg/kg to 10 mg/kg, and a dosage of the ID2 activator is 5 mg/kg to 150 mg/kg.

7. The method of claim 4, wherein the CDK inhibitor is any one or more selected from the group consisting of RO-3306, CGP74514A, BEY-11707, ON-01500, R547, sodium oxamate, dinaciclib (SCH727965), BMS-265246, AZD5438, SU9516, riviciclib hydrochloride (P276-00), AT7519, and NU6027.

8. The method of claim 4, wherein the ID2 activator is any one or more selected from the group consisting of apigenin, isoliquiritigenin, 4-hydroxychalcone, diosmetin, biochanin A, and luteolin.


SEQUENCE LISTING (11 sequences)

SEQ ID NO: 1 - DNA, 1440 bp, synthetic construct
  misc_feature 1..1440: note = TFCP2L1 ORF
  source 1..1440: mol_type = other DNA
atgctcttctggcacacgcagcccgagcactacaaccagcacaactccggcagctacctgcgtgatgtgctcgctctgcccatcttcaagcaggaggaaccccagctgtcccccgagaacgaggcccgcctgccacccctgcaatatgtgttgtgtgctgccacgtccccagccgtgaagctgcatgaagagacgctgacctacctcaaccaaggtcagtcttatgaaatccgactactggagaatcggaagctgggagactttcaagatctgaacacaaaatatgtcaagagcatcatccgtgtggtcttccatgaccgccggctgcagtatacggagcaccagcagctggagggctggcggtggagtcggccaggggaccggatcctggacatcgatattccactgtctgttggtatcttggaccccagggccagcccgacccagctgaatgcagtcgagtttttgtgggaccctgcgaagagagcttctgcattcattcaggtacactgcatcagcacagaattcacccccaggaagcacgggggcgagaagggagtgccctttcgagtccagattgacacgtttaagcagaacgagaatggggagtacacggagcacctgcactcagccagctgccagatcaaggtgttcaagccgaagggagccgatcggaaacagaagactgaccgggagaagatggagaaaagaactgcccaagagaaggagaaataccagccgtcctatgaaaccaccatcctcacagagtgctctccatggcccgacgtggcctaccaggtgaacagcgccccgtccccaagctacaatggttctccaaacagctttggcctcggcgaaggcaacgcctctccgacccacccggtggaggccctgcccgtgggcagtgaccacctgctcccatcagcttcgatccaggatgcccagcagtggcttcaccgcaacaggttctcgcagttctgccggctctttgccagcttctcaggtgctgacttgctgaagatgtcccgagatgatttggtccagatctgtggtcccgcagatgggatccggctcttcaacgccatcaaaggccggaatgtgaggccaaagatgaccatttatgtctgtcaggagctggagcagaatcgagtgcccctgcagcagaagcgggacggcagtggagacagcaacctgtctgtgtaccacgccatcttcctggaagagctgaccaccttggagctgattgagaagatcgccaacctgtacagcatctccccccagcacatccaccgagtctaccggcagggccccacgggcatccatgtggtggtgagcaacgagatggtgcagaacttccaagatgaatcctgttttgtcctcagcacaattaaagctgagagcaatgatggctaccacatcatcctgaaatgtggactctga

SEQ ID NO: 2 - DNA, 405 bp, synthetic construct
  misc_feature 1..405: note = ID2 ORF
  source 1..405: mol_type = other DNA
atgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggctga

SEQ ID NO: 3 - DNA, 21 bp, synthetic construct
  misc_feature 1..21: note = TFCP2L1
  source 1..21: mol_type = other DNA
gctcttcaacgccatcaaagg

SEQ ID NO: 4 - DNA, 19 bp, synthetic construct
  misc_feature 1..19: note = TFCP2L1-F
  source 1..19: mol_type = other DNA
gctcttcaacgccatcaaa

SEQ ID NO: 5 - DNA, 18 bp, synthetic construct
  misc_feature 1..18: note = TFCP2L1-R
  source 1..18: mol_type = other DNA
caggggcactcgattctg

SEQ ID NO: 6 - DNA, 21 bp, synthetic construct
  misc_feature 1..21: note = ID2-F
  source 1..21: mol_type = other DNA
cagcatcccccagaacaagaa

SEQ ID NO: 7 - DNA, 21 bp, synthetic construct
  misc_feature 1..21: note = ID2-R
  source 1..21: mol_type = other DNA
cgatctgcaggtccaagatgt

SEQ ID NO: 8 - DNA, 20 bp, synthetic construct
  misc_feature 1..20: note = ID2_#1-F
  source 1..20: mol_type = other DNA
ctccgatgggttgcagtgaa

SEQ ID NO: 9 - DNA, 23 bp, synthetic construct
  misc_feature 1..23: note = ID2_#1-R
  source 1..23: mol_type = other DNA
cggcagctctaaaatcacagcta

SEQ ID NO: 10 - DNA, 22 bp, synthetic construct
  misc_feature 1..22: note = ID2_#2-F
  source 1..22: mol_type = other DNA
tgcagcacgtcatcgactacat

SEQ ID NO: 11 - DNA, 20 bp, synthetic construct
  misc_feature 1..20: note = ID2_#2-R
  source 1..20: mol_type = other DNA
ctggtgatgcaggctgacaa